(19) United States
(12) Patent Application Publication
(10) Pub. No.: US 2020/0039732 A1
(43) Pub. Date: Feb. 6, 2020

(54) DEVICES FOR DISPENSING A HYDROFLUOROOLEFIN VAPOCOOLANT COMPOSITION AS AN AEROSOL

(71) Applicant: GEBAUER COMPANY, CLEVELAND, OH (US)

(72) Inventors: ANDREW DITTO, HUDSON, OH (US); MONICA VICAREL, RAVENNA, OH (US); RYAN SCAVINSKI, WADSWORTH, OH (US); MARC KRUZER, LYNDHURST, OH (US); JAMES HLEBOVY, CHAGRIN FALLS, OH (US)

(21) Appl. No.: 16/342,712

(22) PCT Filed: Oct. 20, 2017
(86) PCT No.: PCT/US2017/057545

§ 371 Date: Apr. 17, 2019

Related U.S. Application Data
(60) Provisional application No. 62/411,263, filed on Oct. 21, 2016.

(51) Int. Cl. B65D 83/14; B65D 83/20; B65D 83/34; B65D 83/44

(52) U.S. Cl. CPC B65D 83/752; B65D 83/207; A61K 9/124; B65D 83/44; B65D 83/345

(57) ABSTRACT

A device for dispensing a hydrofluoroolefin vapocoolant composition as an aerosol is disclosed. The device comprises an aerosol-dispensing container comprising an internal chamber, a valve, a dispensing hole, and an actuator. The device also comprises a hydrofluoroolefin vapocoolant composition comprising HCFO-1233zd at 70% to 100% (weight/weight) and, optionally, HFO-1234ze in at 0% to 30% (weight/weight). The hydrofluoroolefin vapocoolant composition is stored within the internal chamber. The aerosol-dispensing container is configured so that depressing the actuator reversibly opens the valve to allow the hydrofluoroolefin vapocoolant composition to flow from the internal chamber, past the valve, and through the dispensing hole, thereby dispensing the hydrofluoroolefin vapocoolant composition from the aerosol-dispensing container. A method of treating pain and/or swelling of a tissue of a patient by use of the device also is disclosed. The method comprises a step of dispensing the hydrofluoroolefin vapocoolant composition from the device onto the tissue of the patient.

### FIELD OF THE INVENTION

This application relates generally to devices for dispensing a hydrofluoroolefin vapocoolant composition, and more particularly to devices for dispensing a hydrofluoroolefin vapocoolant composition as an aerosol, the devices comprising: an aerosol-dispensing container comprising an internal chamber, a valve, a dispensing hole, and an actuator; and a hydrofluoroolefin vapocoolant composition comprising trans-1-chloro-3,3,3-trifluoropropene (HCFO-1233zd) at 70% to 100% (weight/weight) and, optionally, trans-1,3,3,3-tetrafluoroprop-1-ene (HFO-1234ze) at 0% to 30% (weight/weight); wherein the hydrofluoroolefin vapocoolant composition is stored within the internal chamber, and the aerosol-dispensing container is configured so that depressing the actuator reversibly opens the valve to allow the hydrofluoroolefin vapocoolant composition to flow from the internal chamber, past the valve, and through the dispensing hole, thereby dispensing the hydrofluoroolefin vapocoolant composition from the aerosol-dispensing container.

### BACKGROUND OF THE INVENTION

Vapocoolant compositions manage pain by evaporating at low boiling point temperatures to achieve a cooling effect. The evaporation requires a phase transition, which removes heat from living tissues, thereby lowering the surface temperature of the tissues. This lowering of temperature decreases the perception of pain.

Previous vapocoolant compositions comprise vapocoolant compounds, such as chlorofluorocarbons (CFC), hydrocarbons, chlorinated hydrocarbons, hydrochlorofluorocarbons (HCFC), and hydrofluorocarbons (HFC), including for example isobutane, butane, isopentane, 1,1,1,2-tetrafluoroethane (HFC-134a), 1,1,1,3,3-pentafluoropropane (HFC-245fa), and chloroethane (ethyl chloride). Such vapocoolant compositions present unfavorable environmental risks, such as high global warming potentials, high ozone depletion potentials, and high maximum incremental reactivities. Furthermore, vapocoolant compositions including vapocoolant compounds and/or blends thereof can exhibit boiling points, vapor pressures, flammabilities, and toxicities that can pose safety risks if used for their cooling effect on living tissues.

Accordingly, a need exists for vapocoolant compositions that can be used to manage pain and swelling without presenting the unfavorable environmental risks and safety risks associated with previous vapocoolant compositions.

### BRIEF SUMMARY OF THE INVENTION

A device for dispensing a hydrofluoroolefin vapocoolant composition as an aerosol is provided. The device comprises an aerosol-dispensing container comprising an internal chamber, a valve, a dispensing hole, and an actuator. The device also comprises a hydrofluoroolefin vapocoolant composition comprising trans-1-chloro-3,3,3-trifluoropropene (HCFO-1233zd) at 70% to 100% (weight/weight) and, optionally, trans-1,3,3,3-tetrafluoroprop-1-ene (HFO-1234ze) at 0% to 30% (weight/weight). The hydrofluoroolefin vapocoolant composition is stored within the internal chamber. The aerosol-dispensing container is configured so that depressing the actuator reversibly opens the valve to allow the hydrofluoroolefin vapocoolant composition to flow from the internal chamber, past the valve, and through the dispensing hole, thereby dispensing the hydrofluoroolefin vapocoolant composition from the aerosol-dispensing container.

In some examples of the device, the hydrofluoroolefin vapocoolant composition comprises HCFO-1233zd at 70% to 99% (weight/weight) and HFO-1234ze at 1 to 30% (weight/weight). In some of these examples, the hydrofluoroolefin vapocoolant composition consists essentially of HCFO-1233zd and HFO-1234ze. Also in some of these examples, the hydrofluoroolefin vapocoolant composition comprises no further vapocoolant compounds.

In some examples of the device, the hydrofluoroolefin vapocoolant composition comprises HCFO-1233zd at 75% to 95% (weight/weight) and HFO-1234ze at 5 to 25% (weight/weight).

In some examples of the device, the aerosol-dispensing container is selected from the group consisting of a stainless steel aerosol can, an aluminum aerosol can, a plastic aerosol can, a polymer-based aerosol can, an aerosol bottle, a glass aerosol bottle, and a pressure container.

In some examples of the device, the aerosol-dispensing container is configured to withstand pressures up to 300 psi (2.07 MPa).

In some examples of the device, the actuator comprises an orifice having a diameter of 0.007 to 0.016 inches (0.18 to 0.40 mm).

In some examples of the device, the actuator is configured for dispensing the hydrofluoroolefin vapocoolant composition without breaking up flow of the hydrofluoroolefin vapocoolant composition.

In some examples of the device, the actuator comprises a non-mechanical break up (NMBU) actuator.

A method of treating pain and/or swelling of a tissue of a patient by use of the device for dispensing a hydrofluoroolefin vapocoolant composition as an aerosol also is provided. The device comprises an aerosol-dispensing container comprising an internal chamber, a valve, a dispensing hole, and an actuator. The device also comprises a hydrofluoroolefin vapocoolant composition comprising HCFO-1233zd at 70% to 100% (weight/weight) and, optionally, HFO-1234ze at 0% to 30% (weight/weight). The hydrofluoroolefin vapocoolant composition is stored within the internal chamber. The aerosol-dispensing container is configured so that depressing the actuator reversibly opens the valve to allow the hydrofluoroolefin vapocoolant composition to flow from the internal chamber, past the valve, and through the dispensing hole, thereby dispensing the hydrofluoroolefin vapocoolant composition from the aerosol-dispensing container. The method comprises a step of dispensing the hydrofluoroolefin vapocoolant composition from the device onto the tissue of the patient.

In some examples of the method, the patient is a human or an animal.

In some examples of the method, the treating comprises managing pain and/or swelling, controlling pain and/or swelling, and/or relieving pain and/or swelling.

In some examples of the method, the dispensing of the hydrofluoroolefin vapocoolant composition from the device onto the tissue of the patient comprises applying the hydrofluoroolefin vapocoolant composition to the tissue of the patient directly by spray and/or indirectly through an applicator.

In some examples of the method, the tissue comprises at least one of intact skin tissue, intact mucous membrane tissue, oral cavity tissue, nasal passage tissue, lip tissue, or minor open wound tissue.

In some examples of the method, the pain and/or swelling of the tissue is due to at least one of a needle procedure, venipuncture, an intravenous catheter start, a cosmetic procedure, a procedure comprising incision and drainage of a small abscess, a minor surgical procedure, a procedure comprising lancing of a boil, a suturing procedure, a stapling procedure, a dental procedure, muscle pain, a scrape, an abrasion, myofascial pain, a hair removal procedure, a tattoo procedure, a medical tattoo procedure, chronic pain, viral infection pain, herpes sore pain, and a bruise. Alternatively or additionally, in some examples of the method, the pain and/or swelling of the tissue is due to at least one of swelling from a sports injury, swelling from overuse of a muscle, swelling from overuse of a joint, and swelling from arthritis. Alternatively or additionally, in some examples of the method, the actuator of the device is positioned 2 to 20 inches (5 to 51 cm) from the tissue during the step of dispensing. Alternatively or additionally, in some examples of the method, the step of dispensing is carried out for 2 to 15 seconds.

In some examples of the method, the treating comprises use of the hydrofluoroolefin vapocoolant composition as a counterirritant in the management of at least one of myofascial pain, restricted motion, or muscle tension. Alternatively or additionally, in some examples of the method, the actuator of the device is positioned 6 to 24 inches (15 to 61 cm) from the tissue during the step of dispensing. Alternatively or additionally, in some examples of the method, the step of dispensing comprises directing a spray of the hydrofluoroolefin vapocoolant composition in parallel sweeps 0.3 to 2 inches (0.8 to 5 cm) apart at a rate of approximately 3 to 5 inches (8 to 13 cm) per second.

In some examples of the method, the treating comprises use of the hydrofluoroolefin vapocoolant composition for identifying pulp viability of teeth.

In some examples of the method, the treating comprises use of the hydrofluoroolefin vapocoolant composition for tracking the efficacy of an epidural block.

In some examples of the method, the treating has no toxic or irritating effect on a living tissue of the patient.

### BRIEF DESCRIPTION OF THE DRAWINGS

FIG. 1 shows an embodiment of a device for dispensing a hydrofluoroolefin vapocoolant composition as an aerosol, in perspective view.

FIG. 2 shows the device of FIG. 1, in front view.

FIG. 3 shows the device of FIG. 1, in side view.

FIG. 4 shows the device of FIG. 1, in side sectional view.

FIG. 5 shows the structure of trans-1-chloro-3,3,3-trifluoropropene, also termed HCFO-1233zd.

FIG. 6 shows the structure of trans-1,3,3,3-tetrafluoroprop-1-ene, also termed HFO-1234ze or trans-1,3,3,3-tetrafluoropropene.

FIG. 7 shows an MBU actuator insert, in perspective view.

FIG. 8 shows the MBU actuator insert of FIG. 7, in side view.

FIG. 9 shows the MBU actuator insert of FIG. 7, in sectional view.

FIG. 10 shows an NMBU actuator insert, in perspective view.

FIG. 11 shows the NMBU actuator insert of FIG. 10, in side view.

FIG. 12 shows the NMBU actuator insert of FIG. 10, in sectional view.

FIG. 13 shows a plot of minimum cooling temperature with various hydrofluoroolefin vapocoolant compositions of HCFO-1233zd and HFO-1234ze administered from an aerosol can with a medium stream nozzle from various distances.

FIG. 14 shows a plot of cooling temperature with various hydrofluoroolefin vapocoolant compositions of HCFO-1233zd and HFO-1234ze administered from an aerosol can with a fine stream nozzle from various distances.

FIG. 15 shows a plot of minimum cooling profile with various hydrofluoroolefin vapocoolant compositions of HCFO-1233zd and HFO-1234ze administered from an aerosol can with a mist spray nozzle from various distances.

### DETAILED DESCRIPTION

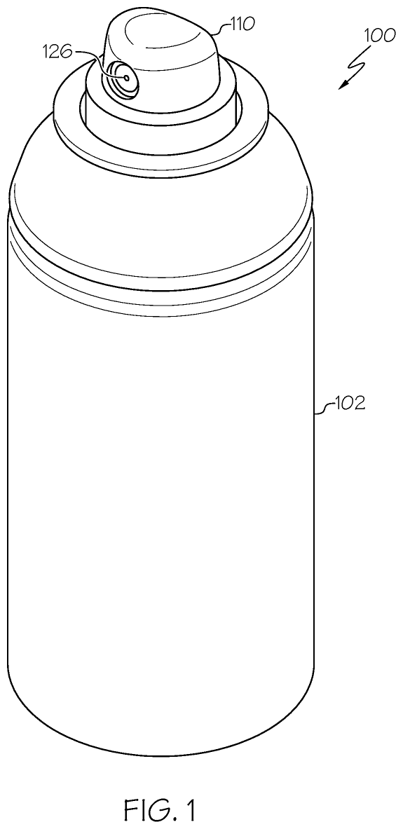

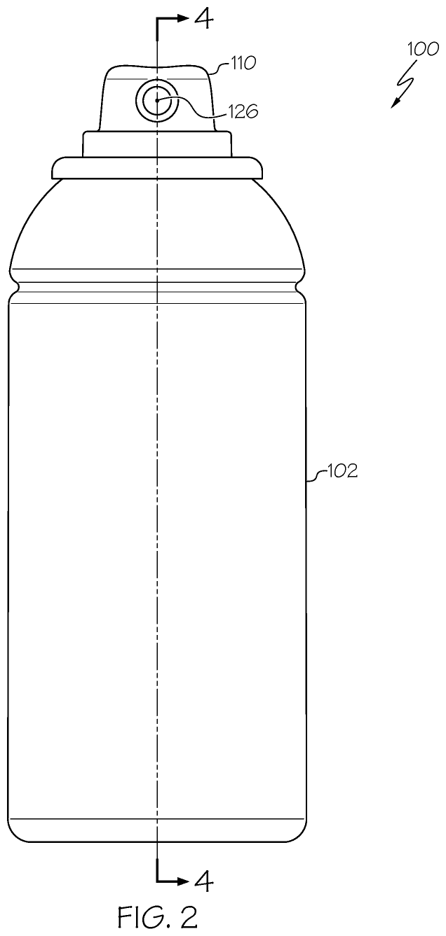

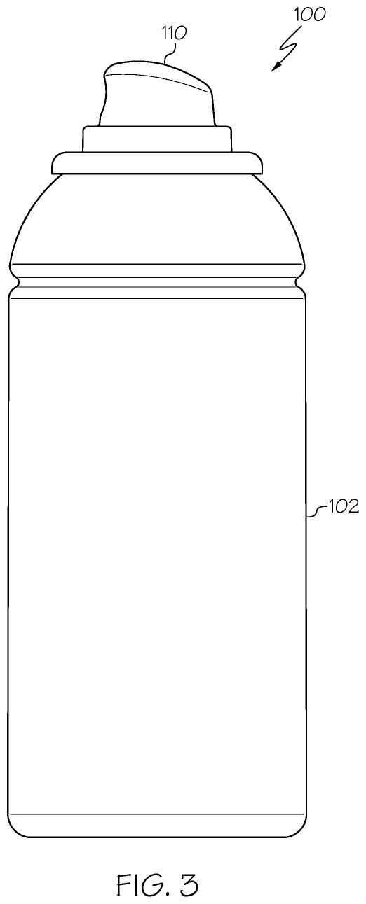

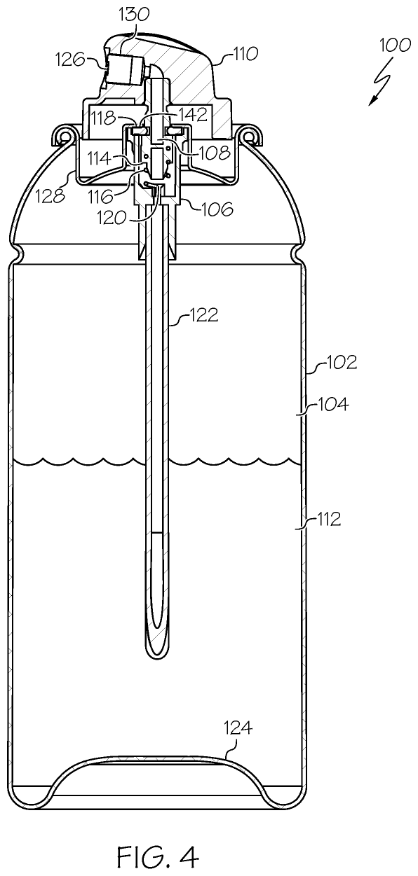

As shown in FIG. 1, FIG. 2, FIG. 3, and FIG. 4, a device 100 for dispensing a hydrofluoroolefin vapocoolant composition as an aerosol is disclosed. As shown in FIG. 4, the device 100 comprises an aerosol-dispensing container 102 comprising an internal chamber 104, a valve 106, a dispensing hole 108, and an actuator 110. The device 100 also comprises a hydrofluoroolefin vapocoolant composition 112 comprising trans-1-chloro-3,3,3-trifluoropropene (HCFO-1233zd) at 70% to 100% (weight/weight) and, optionally, trans-1,3,3,3-tetrafluoroprop-1-ene (HFO-1234ze) at 0% to 30% (weight/weight). The hydrofluoroolefin vapocoolant composition 112 is stored within the internal chamber 104. The aerosol-dispensing container 102 is configured so that depressing the actuator 110 reversibly opens the valve 106 to allow the hydrofluoroolefin vapocoolant composition 112 to flow from the internal chamber 104, past the valve 106, and through the dispensing hole 108, thereby dispensing the hydrofluoroolefin vapocoolant composition 112 from the aerosol-dispensing container 102.

Without wishing to be bound by theory, it is believed that HCFO-1233zd unexpectedly provides advantageous properties as a vapocoolant composition when dispensed as an aerosol, for treating pain and/or swelling, without presenting the unfavorable environmental risks and safety risks associated with previous vapocoolant compositions. Moreover, it is believed that hydrofluoroolefin vapocoolant compositions comprising HCFO-1233zd at 70% to 100% (weight/weight) and HFO-1234ze at up to 30% (weight/weight) unexpectedly provide advantageous composition properties when dispensed as an aerosol, again for treating pain and/or swelling, without presenting the unfavorable environmental risks and safety risks associated with previous vapocoolant compositions, that would not have been predictable based on properties of HCFO-1233zd alone or HFO-1234ze alone, nor achievable based on other ratios of the hydrofluoroolefins or combinations of other hydrofluoroolefins, HFCs, HCFCs, CFCs, hydrocarbons, or chlorinated hydrocarbons. Despite differences in various properties, such as surface tension and liquid viscosity, of each of HCFO-1233zd and HFO-1234ze, in comparison to HFC-245fa, HFC-134a, and other previous vapocoolant compounds (TABLE 1), it has been determined that hydrofluoroolefin vapocoolant compositions comprising HCFO-1233zd at 70% to 100% (weight/weight) and HFO-1234ze at up to 30% (weight/weight) can provide vapor pressures at safe levels for containment in aerosol-dispensing containers for use in pain and swelling management for human and animal patients (TABLE 2). Also it has been determined that hydrofluoroolefin vapocoolant compositions comprising HCFO-1233zd at 70% to 100% (weight/weight) and HFO-1234ze at up to 30% (weight/weight) can provide minimum cooling temperatures at various spray distances and dispense rates indicating safe levels of cooling appropriate for use on living tissues as a vapocoolant to treat pain and/or swelling (TABLE 3 and TABLE 4, respectively).

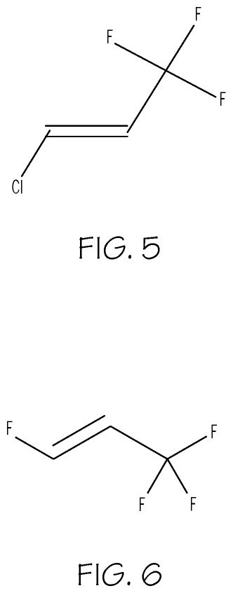

As noted, the hydrofluoroolefin vapocoolant composition 112 comprises HCFO-1233zd at 70% to 100% (weight/weight) and, optionally, HFO-1234ze at 0% to 30% (weight/weight). The structures of HCFO-1233zd and HFO-1234ze are shown in FIG. 5 and FIG. 6, respectively. HCFO-1233zd is available commercially, for example as Honeywell SOLSTICE® 1233zd(E) product. HFO-1234ze also is available commercially, for example as Honeywell HFO-1234ze blowing agent product. In some examples the hydrofluoroolefin vapocoolant composition 112 can comprise HCFO-1233zd at 70% to 99% (weight/weight) and HFO-1234ze at 1 to 30% (weight/weight), i.e. the hydrofluoroolefin vapocoolant composition 112 can comprise both HCFO-1233zd and HFO-1234ze, each in the amounts recited respectively. In some examples the hydrofluoroolefin vapocoolant composition 112 can consist essentially of HCFO-1233zd and HFO-1234ze, i.e. the hydrofluoroolefin vapocoolant composition 112 can include both HCFO-1233zd and HFO-1234ze, and potentially additional compounds, but not additional compounds that would materially affect the vapocoolant characteristics of the hydrofluoroolefin vapocoolant composition 112. In some examples the hydrofluoroolefin vapocoolant composition 112 comprises no further vapocoolant compounds.

In some examples the hydrofluoroolefin vapocoolant composition 112 comprises HCFO-1233zd at 75% to 95% (weight/weight) and HFO-1234ze at 5 to 25% (weight/weight). For example, the hydrofluoroolefin vapocoolant composition 112 can comprise HCFO-1233zd at about 75% (weight/weight) and HFO-1234ze at about 25% (weight/weight) (e.g. 73% to 77% and 23% to 27%, respectively, or 74% to 76% and 24% to 26%, respectively, or 75% and 25%, respectively). Also for example, the hydrofluoroolefin vapocoolant composition 112 can comprise HCFO-1233zd at about 80% (weight/weight) (e.g. 78% to 82%, or 79% to 81%, or 80%), about 85% (weight/weight) (e.g. 83% to 87%, or 84% to 86%, or 85%), or about 90% (weight/weight) (e.g. 88% to 92%, or 89% to 91%, or 90%) and HFO-1234ze at about 20% (weight/weight) (e.g. 18% to 22%, or 19% to 21%, or 20%), about 15% (weight/weight) (e.g. 13% to 17%, or 14% to 16%, or 15%), or about 10% (weight/weight) (e.g. 8% to 12%, or 9% to 11%, or 10%), respectively.

The aerosol-dispensing container 102 can be an aerosol can, an aerosol bottle, or other suitable aerosol-dispensing container. For example, the aerosol-dispensing container 102 can be selected from the group consisting of a stainless steel aerosol can, an aluminum aerosol can including an epoxy phenolic lining, a plastic aerosol can, a polymer-based aerosol can, an aerosol bottle, a glass aerosol bottle, and a pressure container. Also for example, the aerosol-dispensing container 102 can be configured to withstand pressures up to 300 psi (2.07 MPa). Regarding the aerosol can in particular, the aerosol can may be, for example, a three piece can, a two piece can, a United States Department of Transportation (DOT) rated can, such as a DOT-2P can for internal pressures up to 160 psi (1.10 MPa) at 130° F. (54.4° C.), or a DOT-2Q can for internal pressures up to 180 psi (1.24 psi) at 130° F. (54.4° C.), or a special pressure rated can.

As noted, the aerosol-dispensing container 102 is configured so that depressing the actuator 110 reversibly opens the valve 106 to allow the hydrofluoroolefin vapocoolant composition 112 to flow from the internal chamber 104, through the valve 106, and through the dispensing hole 108, thereby dispensing the hydrofluoroolefin vapocoolant composition 112 from the aerosol-dispensing container 102. As shown in FIG. 4, in some examples, the valve 106 can comprise a spring cup 114, a spring 116, a gasket 118, and a vapor tap 120. Also, the valve 106 can have a dip tube 122 attached thereto, and the internal chamber 104 of the aerosol-dispensing container 102 can comprise a lower end 124, such that the dip tube 122 extends into the internal chamber 104 of the aerosol-dispensing container 102, toward the lower end 124 of the internal chamber 104. The dip tube 122 is open at both of its ends. In accordance with these examples, depressing the actuator 110 can displace the spring cup 114 and compress the spring 116, thereby opening the valve 106, allowing the hydrofluoroolefin vapocoolant composition 112 to flow from the internal chamber 104, past the valve 106, e.g. around and/or through the valve 106, and through the dispensing hole 108. In accordance with these examples, upon release of the actuator 110, the spring 116 can extend, returning the spring cup 114 to its non-displaced position, thereby closing the valve 106, preventing the hydrofluoroolefin vapocoolant composition 112 from flowing from the internal chamber 104. The gasket 118 on the valve 106 can provide a seal in the valve 106 to prevent outflow of the hydrofluoroolefin vapocoolant composition 112 from the internal chamber 104 when the valve 106 is closed. The vapor tap 120 on the valve 106 can provide additional propellant force and control of mist particle size. The dip tube 122 attached to the valve 106 can allow the hydrofluoroolefin vapocoolant composition 112 on the lower end 124 of the internal chamber 104 to flow through the valve 106. Other suitable configurations, for example as described above except without a vapor tap 120 and/or without a dip tube 122, among other suitable configurations, also can also be used.

The valve 106 can be, for example, a vertical valve that opens with vertical pressure on the valve, a tilt valve that opens with forward pressure on the valve, an “up/down” valve that can dispense in an upright or inverted position, a female valve that interfaces with a stem in the actuator 110, a male valve that interfaces with a slot or a channel in the actuator 110, a bag on valve that contains bag contents that do not mix with propellants, a metering valve that dispenses an exact amount of contents, a high delivery valve, or a variable valve for multiple discharges, among other valves.

The dip tube 122 can be made from, for example, low density polyethylene (LDPE), medium density polyethylene (MDPE), polypropylene, polytetrafluoroethylene (Teflon), among other suitable materials that are compatible with the hydrofluoroolefin vapocoolant composition.

The gasket 118 can be, for example, an outer gasket. For example, as shown in FIG. 4, in some examples the device 100 includes a valve cup 128. In accordance with these examples, the gasket 118 can be an outer gasket that forms a seal between the valve cup 128 and the aerosol-dispensing container 102. The gasket 118 also can be, for example, an inner gasket 142 that covers the dispensing hole in the valve. The gasket 118 can be made, for example, from buna, chlorobutyl, neoprene, butyl, or a fluoroelastomer, such as ZYTEL fluoroelastomer, among other materials.

The actuator 110 can be one that is suitable for allowing the hydrofluoroolefin vapocoolant composition 112, during dispensing from the aerosol-dispensing container 102, to reach a tissue of a patient, before the hydrofluoroolefin vapocoolant composition 112 has mostly or entirely evaporated. For example, the actuator 110 can comprise an actuator insert 130 comprising an orifice 126 having a diameter of 0.007 to 0.016 inches (0.18 to 0.40 mm). Also for example, the actuator 110 can be configured for dispensing the hydrofluoroolefin vapocoolant composition 112 without breaking up flow of the hydrofluoroolefin vapocoolant composition 112, e.g. based on the actuator insert 130 comprising a smooth bore. Also for example, the actuator 110 can comprise a non-mechanical break up (NMBU) actuator, e.g. again based on the actuator insert 130 comprising a smooth bore.

Without wishing to be bound by theory, it is believed that use of an actuator 110 that comprises an actuator insert 130 comprising an orifice 126 having a diameter of 0.007 to 0.016 inches (0.18 to 0.40 mm), that is configured for dispensing the hydrofluoroolefin vapocoolant composition without breaking up flow of the hydrofluoroolefin vapocoolant composition, and/or that comprises a non-mechanical break up (NMBU) actuator, unexpectedly provides advantages by promoting formation of mist particles of the hydrofluoroolefin vapocoolant compositions that do not mostly or entirely evaporate before reaching the tissue of a patient.

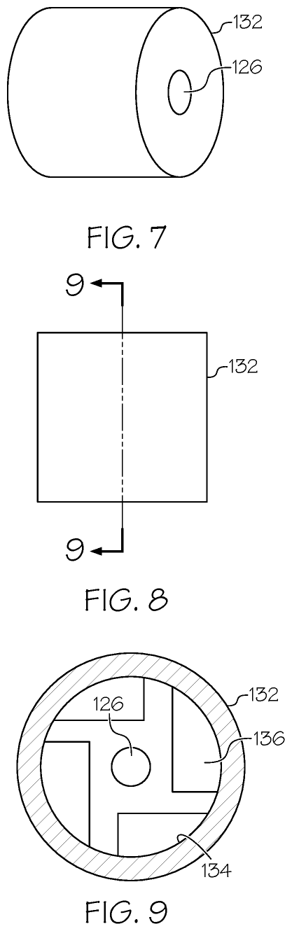

The configuration of the actuator 110 affects the minimum cooling temperature of the tissue (TABLE 3). Specifically, use of a mechanical break up (MBU) actuator for misting of the hydrofluoroolefin vapocoolant compositions can result in failure to achieve a minimum cooling temperature associated with an effective vapocoolant (−15° C. to 15° C.), even at relatively close spray distances, e.g. 5 inches (13 cm). As shown in FIG. 7, FIG. 8, and FIG. 9, MBU actuators are designed to include an actuator insert 130 that is an MBU actuator insert 132, typically made from plastic, that is located within the actuator 110 and that generates a swirling or misting spray pattern. The MBU actuator insert 132 includes an internal surface 134 and two to four offset radial channels 136 on the internal surface 134. The two to four offset radial channels 136 act to cause a strong swirling action in the hydrofluoroolefin vapocoolant composition 112 flowing therethrough, as the hydrofluoroolefin vapocoolant composition 112 leaves the MBU actuator insert 132 and the actuator 110. This swirling action can lead to droplet formation and a misting spray.

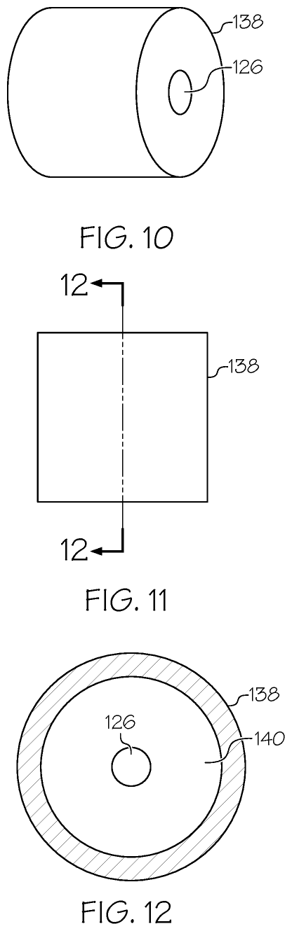

In contrast, use of an NMBU actuator can achieve a minimum cooling temperature associated with an effective vapocoolant, with respect to both stream and mist actuators (TABLE 3). The use of an NMBU actuator also results in the hydrofluoroolefin vapocoolant compositions having dispensing rates more similar to those of control vapocoolant compositions comprising HFCs, particularly for hydrofluoroolefin vapocoolant compositions comprising increasing amounts of HCFO-1233zd (TABLE 4). As shown in FIG. 10, FIG. 11, and FIG. 12, NMBU actuators are designed to include an actuator insert 130 that is an NMBU actuator insert 138 that generates a more laminar flow spray in comparison to an MBU actuator insert 132. An NMBU actuator insert 138 is located within the actuator 110 and contains a smooth bore channel 140 that leads to a more laminar flow.

A method of treating pain and/or swelling of a tissue of a patient by use of the device 100 for dispensing a hydrofluoroolefin vapocoolant composition as an aerosol also is disclosed.

The device 100 is as described above. Accordingly, the device 100 comprises an aerosol-dispensing container 102 comprising an internal chamber 104, a valve 106, a dispensing hole 108, and an actuator 110. The device 100 also comprises a hydrofluoroolefin vapocoolant composition 112 comprising HCFO-1233zd at 70% to 100% (weight/weight) and, optionally, HFO-1234ze at 0% to 30% (weight/weight). The hydrofluoroolefin vapocoolant composition 112 is stored within the internal chamber 104. The aerosol-dispensing container 102 is configured so that depressing the actuator 110 reversibly opens the valve 106 to allow the hydrofluoroolefin vapocoolant composition 112 to flow from the internal chamber 104, past the valve 106, and through the dispensing hole 108, thereby dispensing the hydrofluoroolefin vapocoolant composition 112 from the aerosol-dispensing container 102.

The method comprises a step of dispensing the hydrofluoroolefin vapocoolant composition 112 from the device 100 onto the tissue of the patient. The patient can be a human or an animal. For example, the patient can be a human in need of treatment for pain and/or swelling. Also for example, the patient can be an animal in need of treatment for pain and/or swelling.

In some examples, the treating comprises managing pain and/or swelling, controlling pain and/or swelling, and/or relieving pain and/or swelling.

In some examples, the dispensing of the hydrofluoroolefin vapocoolant composition 112 from the device onto the tissue of the patient comprises applying the hydrofluoroolefin vapocoolant composition 112 to the tissue of the patient directly by spray and/or indirectly through an applicator. Regarding applying the hydrofluoroolefin vapocoolant composition 112 indirectly through an applicator, the applicator can be, for example, gauze, a cotton swab, a sponge, a straw, or another similar applicator.

In some examples, the tissue comprises at least one of intact skin tissue, intact mucous membrane tissue, oral cavity tissue, nasal passage tissue, lip tissue, or minor open wound tissue.

In some of these examples, the pain and/or swelling of the tissue is due to at least one of a needle procedure, venipuncture, an intravenous catheter start, a cosmetic procedure, a procedure comprising incision and drainage of a small abscess, a minor surgical procedure, a procedure comprising lancing of a boil, a suturing procedure, a stapling procedure, a dental procedure, muscle pain, a scrape, an abrasion, myofascial pain, a hair removal procedure, a tattoo procedure, a medical tattoo procedure, chronic pain, viral infection pain, herpes sore pain, and a bruise.

Also in some of these examples, the pain and/or swelling of the tissue is due to at least one of swelling from a sports injury, swelling from overuse of a muscle, swelling from overuse of a joint, and swelling from arthritis.

Also in some of these examples the actuator 110 of the device 100 is positioned 2 to 20 inches (5 to 51 cm) from the tissue during the step of dispensing. For example, the actuator 110 of the device 100 can be positioned 2.5 to 12 inches (6 to 31 cm) or 3 to 7 inches (8 to 18 cm) from the tissue during the step of dispensing.

Also in some of these examples the step of dispensing is carried out for 2 to 15 seconds. For example, the step of dispensing can be carried out for 3 to 12 seconds or 4 to 10 seconds.

As noted above, in some examples the tissue comprises at least one of intact skin tissue, intact mucous membrane tissue, oral cavity tissue, nasal passage tissue, lip tissue, or minor open wound tissue.

In some of these examples, the treating comprises use of the hydrofluoroolefin vapocoolant composition 112 as a counterirritant in the management of at least one of myofascial pain, restricted motion, or muscle tension.

Also in some of these examples, the actuator 110 of the device 100 is positioned 6 to 24 inches (15 to 61 cm) from the tissue during the step of dispensing. For example, the actuator 110 of the device 100 can be positioned 9 to 21 inches (23 to 53 cm) or 12 to 18 inches (30 to 46 cm) from the tissue during the step of dispensing.

Also in some of these examples, the step of dispensing comprises directing a spray of the hydrofluoroolefin vapocoolant composition 112 in parallel sweeps 0.3 to 2 inches (0.8 to 5 cm) apart at a rate of approximately 3 to 5 inches (8 to 13 cm) per second. For example, the step of dispensing can comprise directing a spray of the hydrofluoroolefin vapocoolant composition 112 in parallel sweeps 0.4 to 1.5 inches (1 to 4 cm) apart at a rate of approximately 3.5 to 4.5 inches (9 to 11 cm) per second. Also for example, the step of dispensing can comprise directing a spray of the hydrofluoroolefin vapocoolant composition 112 in parallel sweeps 0.5 to 1 inches (1.5 to 2 cm) apart at a rate of approximately 4 inches (10 cm) per second.

Again as noted above, in some examples the tissue comprises at least one of intact skin tissue, intact mucous membrane tissue, oral cavity tissue, nasal passage tissue, lip tissue, or minor open wound tissue.

In some of these examples, the treating comprises use of the hydrofluoroolefin vapocoolant composition 112 for identifying pulp viability of teeth.

In some examples, the treating comprises use of the hydrofluoroolefin vapocoolant composition for tracking the efficacy of an epidural block.

In some examples, the treating has no toxic or irritating effect on a living tissue of the patient.

### EXAMPLES

### Example 1: Vapocoolant Compound Properties

Properties of various vapocoolant compounds are provided in TABLE 1.

TABLE 1
Vapocoolant compound properties.

| | HFC- | HFC- | HFO- | HCFO- | Iso- | Ethyl |
|---|---|---|---|---|---|---|
| Properties | 134a | 245fa | 1234ze | 1233zd | Butane | Chloride |
| Molecular Weight (g/mol) | 102.0 | 134.1 | 114 | 130.5 | 58.12 | 64.51 |
| Boiling Point, Tb | −26° C. | 15.3° C. | −19° C. | 19° C. | −11.7° C. | 12.3° C. |
| Critical Point, Tc | 102° C. | 154° C. | 109° C. | >154° C. | 134.7 | 187° C. |
| Pvap, MPa (25° C.) | 0.665 | 0.124 | 0.484 | 0.108 | 0.319 | 0.135 |
| Pvap, MPa (80° C.) | 2.63 | 0.789 | 2.01 | 0.650 | 1.5 | 0.745 |
| Density (g/cm³) | 1.21 | 1.32 | 1.17 | 1.27 | 0.554 | 0.890 |
| Surface Tension (dyne/cm | 8.69 | 13.6 | 13.3 | 14.039 | 10.3 | 19.5 |
| at 20° C.) | | | | | | |
| Liquid Viscosity (cP at 20° C.) | 0.202 | 0.402 | 0.489 | 0.319 | 0.156 | 0.279 |

### Example 2: Internal Pressures

Internal pressures within stream and mist aerosol can configurations for various hydrofluoroolefin vapocoolant compositions comprising HCFO-1233zd/HFO-1234ze were determined.

REFERENCES: Modified T-031.4 USP Pressure Test; USP <601> Aerosols/Physical Tests: Pressure Test.

EQUIPMENT: Water bath capable of maintaining a temperature of 25° C. (+/−3° C.); Pressure gauge.

TEST SAMPLES—MATERIAL: 2(n=9) HCFO-1233zd 100%; 2(n=9) HCFO-1233zd 95%/HFO-1234ze 5%; 2(n=9) HCFO-1233zd 90%/HFO-1234ze 10%; 2(n=9) HCFO-1233zd 80%/HFO-1234ze 20%; 2(n=9) HCFO-1233zd 70%/HFO-1234ze 30% (referred to as 70%). HCFO-1233zd corresponded to Honeywell SOLSTICE® 1233zd(E). HFO-1234ze corresponded to Honeywell HFO-1234ze blowing agent.

TEST SAMPLES—COMPONENTS: 2(n=15) Medium Stream can; 2(n=15) Fine Stream can; 2(n=15) Mist can.

TEST SAMPLES—HFC CONTROLS: (n=4) Gebauer's PAIN EASE® Mist brand product (lot 1); (n=3) Gebauer's PAIN EASE® Mist brand product (lot 2); (n=4) Gebauer's SPRAY AND STRETCH® Fine Stream brand product (lot 1); (n=3) Gebauer's SPRAY AND STRETCH® Fine Stream brand product (lot 2); (n=7) Gebauer's PAIN EASE® Medium Stream brand product (lot 1).

METHODS: An initial 45 cans were sampled, 15 Medium Stream cans, 15 Fine Stream cans, and 15 Mist cans. All the caps and actuators were removed from each can. The cans were then encased in a plastic sleeve and immersed in a constant-temperature water bath at 25° C. (+/−3° C.) for 30 minutes. The capacity for the water bath allowed 15 cans at a time. Each can was removed from the water bath and dried with a towel. The can was then placed in an upright position on the pressure meter. The pressure reading was taken and recorded. This process was repeated with the controls to determine a baseline. An initial 12 control cans were sampled, 4 HFC Medium Stream cans, 4 HFC Fine Stream cans, and 4 HFC Mist cans. Another batch of testing was performed following the same procedures for a total of 80 cans sampled.

RESULTS: Results are provided in TABLE 2.

TABLE 2
Internal pressures within stream and mist aerosol can configurations for
various hydrofluoroolefin vapocoolant compositions comprising
HCFO-1233zd/HFO-1234ze.

| | 100%/0% | 95%/5% | 90%/10% | 80%/20% | 70%/30% |
|---|---|---|---|---|---|
| Actuator | psi | psi | psi | psi | psi |
| Type | (MPa) | (MPa) | (MPa) | (MPa) | (MPa) |
| NMBU | 23.8 | 21.5 | 19.3 | 25.1 | 30.5 |
| Medium | (0.164) | (0.148) | (0.133) | (0.173) | (0.210) |
| Stream | | | | | |
| NMBU Fine | 28.6 | 32.1 | 19.5 | 26.8 | 29.3 |
| Stream | (0.197) | (0.221) | (0.134) | (0.185) | (0.202) |
| MBU Mist | 24.4 | 20.6 | 23.8 | 25.9 | 30.5 |
| | (0.168) | (0.142) | (0.164) | (0.179) | (0.210) |

DISCUSSION: The results indicate that hydrofluoroolefin vapocoolant compositions comprising HCFO-1233zd at 70% to 100% (weight/weight) and HFO-1234ze at up to 30% (weight/weight) can provide vapor pressures at safe levels for containment in aerosol-dispensing containers for use in treating patients. Medium Stream, Fine Stream, and Mist cans containing the 90% HCFO-1233zd/10% HFO-1234ze formulation yielded pressures most equivalent to those of the HFC controls.

### Example 3: Minimum Cooling Temperatures

Minimum cooling temperatures at various spray distances were determined for NMBU and MBU actuators.

REFERENCES: Modified Design P-29.5 Cooling Effect Testing for Aerosol Skin Refrigerants.

EQUIPMENT: Cooling Effects Device; Personal computer with the Cooling Effect data acquisition program (PLC) and spreadsheet software.

TEST SAMPLES—MATERIAL: 2(n=9) HCFO-1233zd 100%; 2(n=9) HCFO-1233zd 95%/HFO-1234ze 5%; 2(n=9) HCFO-1233zd 90%/HFO-1234ze 10%; 2(n=9) HCFO-1233zd 80%/HFO-1234ze 20%; 2(n=9) HCFO-1233zd 70%/HFO-1234ze 30%. HCFO-1233zd corresponded to Honeywell SOLSTICE® 1233zd(E). HFO-1234ze corresponded to Honeywell HFO-1234ze blowing agent.

TEST SAMPLES—COMPONENTS: 2(n=15) Medium Stream can; 2(n=15) Fine Stream can; 2(n=15) mechanical break up (MBU) Mist can, (n=8) non-mechanical break up (NMBU) 0.016″ Mist can, (n=8) NMBU 0.013″ Mist can.

TEST SAMPLES—HFC CONTROLS: (n=4) Gebauer's PAIN EASE® Mist brand product (lot 1); (n=3) Gebauer's PAIN EASE® Mist brand product (lot 2); (n=4) Gebauer's SPRAY AND STRETCH® Fine Stream brand product (lot 1); (n=3) Gebauer's SPRAY AND STRETCH® Fine Stream brand product (lot 2); (n=7) Gebauer's PAIN EASE® Medium Stream brand product (lot 1).

METHODS: An initial 45 cans were sampled, 15 Medium Stream cans, 15 Fine Stream cans, 15 MBU Mist cans, 8 NMBU 0.016″ Mist cans, and 8 NMBU 0.013″ Mist cans. The Cooling Effects Device was turned on and allowed to warm up. The water bath temperature was checked to ensure it had reached the optimal 33° C. before testing began. The Cooling Effects Device was then programmed to perform timed spray tests, with a spray on time of five (5) seconds, and a spray off time of thirty (30) seconds. The spray count set point was programmed to three (3) so that each sample would need to be actuated three times. The Medium and Fine Stream HFO formulations were tested at 3, 7, and 18 inches (7.6, 18, and 46 cm, respectively). The Mist HFO formulations were tested at 3 and 5 inches (7.6 and 13 cm, respectively).

This process was repeated with the controls to determine a baseline. An initial 12 control cans were sampled, 4 HFC Medium Stream cans, 4 HFC Fine Stream cans, and 4 HFC Mist cans. Another batch of testing was performed following the same procedures for a total of 96 cans sampled and a total of 21 control cans sampled.

All raw data were recorded. The lowest temperature of each spray was determined. The lowest temperatures for the three sprays were then averaged.

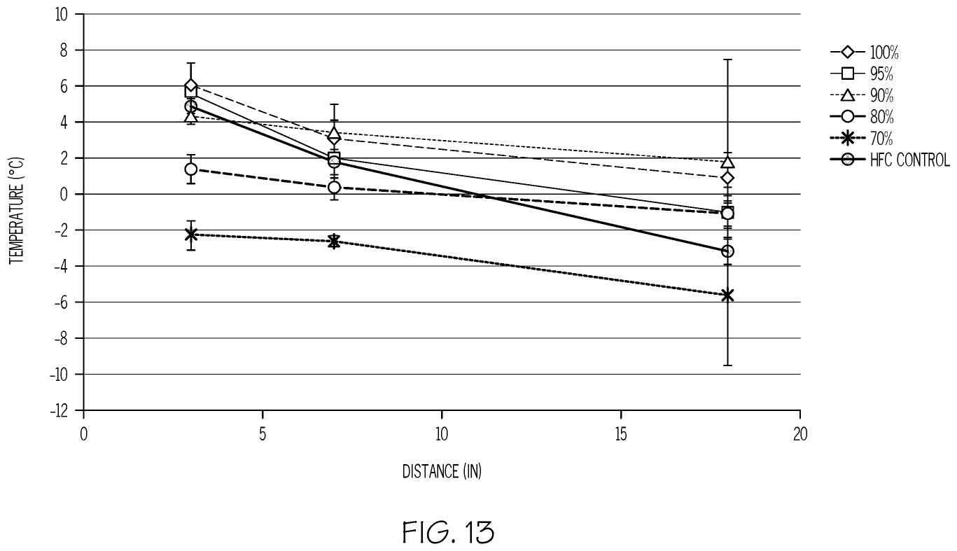

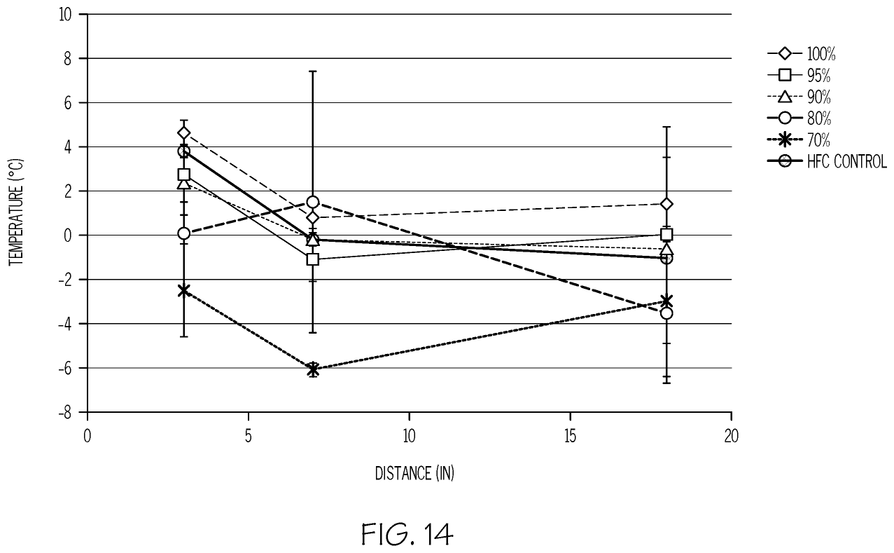

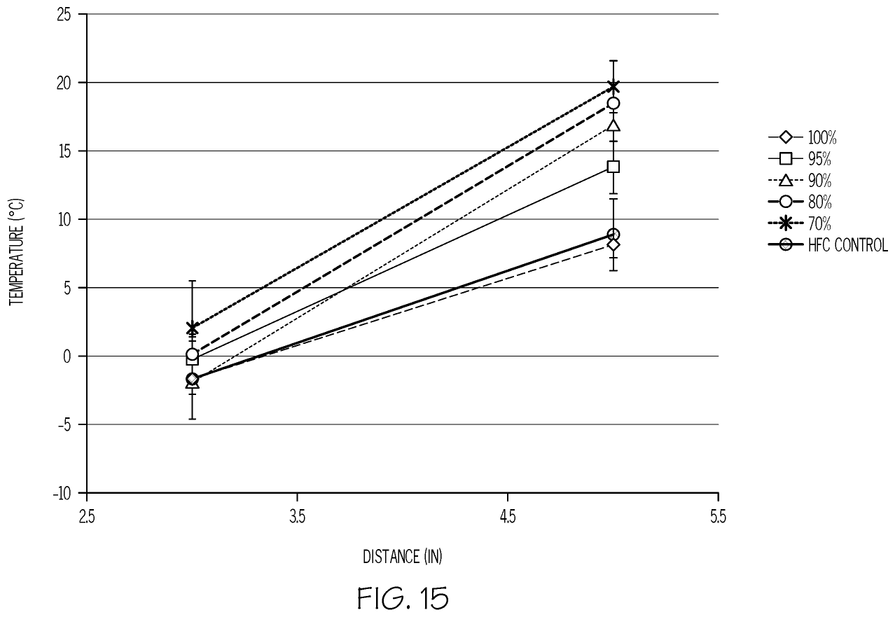

RESULTS: Results are provided in TABLE 3, FIG. 13, FIG. 14, and FIG. 15.

TABLE 3
Aerosol actuator type and the corresponding minimum cooling
temperature for hydrofluoroolefin vapocoolant composition
comprising HCFO-1233zd at 90% (weight/weight) and HFO-1234ze
at 10% (weight/weight), at various spray distances.

| | Spray | |
|---|---|---|
| | Distance | 90%/10% |
| Actuator Type | in | (C.°) |
| NMBU Medium Stream 0.09″ | 3 | 3.7 |
| | 7 | 1.4 |
| | 18 | −1.0 |
| NMBU Fine Stream | 3 | 3.1 |
| 0.007″ | 7 | 1.7 |
| | 18 | 0.0 |
| MBU Mist | 3 | −1.7 |
| 0.016″ | 5 | 16.9 |
| NMBU Course Mist 0.013″ | 3 | −3.4 |
| | 5 | −1.2 |
| NMBU Mist 0.016″ | 3 | −4.5 |
| | 5 | −1.0 |

DISCUSSION: Overall averages of the cooling effects for the HFO Medium and Fine Stream formulations were within specification at all distances (−15° C. to 15° C.). However, the HFO Mist with MBU actuators were within specification only at 3 inches (7.6 cm). Results show similar standard deviations of the Medium, Fine Stream, and Mist with NMBU actuators for HFO formulations compared to the HFC controls. Initial testing revealed inconsistent spraying from the Fine Stream cans. Some observations made included mist-like sprays, sputtering or splitting, or no spray at all. Upon further investigation, it was found that the faulty sprays were due to the actuators. Replacement of these defective actuators resolved the spray issues. A total of 6 Fine Stream cans and 1 Medium Stream can were retested with different actuators. The new data from the testing shows lower temperatures as expected. The purpose of retesting the specific cans was to reduce inaccurate readings because the initial readings were not a true representation of the HFO temperatures due to improper nozzle functionality.

Temperature readings for HFO Mist sprays with MBU actuators are higher than the HFC controls. As the distance increases from 3 inches (7.6 cm) to 5 inches (13 cm), the temperature increases as well. This is likely due to the thermocouple being unable to detect the spray past a certain point. Differences such as surface tension, viscosity, or pressure may be causing the droplet diameter to decrease and to disperse differently.

A second batch of cooling effects testing was performed to increase the sampling size and to validate initial results. The second batch of cans were crimped, vacuumed, and filled within 24 hours to help eliminate the possibility of vacuum loss. The data were more consistent and less variable in comparison to the first batch.

Spray patterns of the Mist Spray taken depict larger diameters for the 100% HCFO-1233zd, 95% HCFO-1233zd/5% HFO-1234ze, and 90% HCFO-1233zd/10% HFO-1234ze formulations at 3 inches (7.6 cm). The 80% HCFO-1233zd/20% HFO-1234ze and 70% HCFO-1233zd/30% HFO-1234ze formulations were much smaller with outer rings of fine droplets. At 5 inches (13 cm), the 100%, 95%, and 90% formulations had larger diameters than the controls. The 80% and 70% formulations had smaller spray diameters than the controls.

All of the cooling effects temperatures of the HFO Medium and Fine Stream sprays were within specification. Data values similar to those of the HFC controls were found in the 95% HCFO-1233zd/5% HFO-1234ze and 90% HCFO-1233zd/10% HFO-1234ze formulations. After completing another batch of testing, the formulation that is most equivalent was determined to be the 90% HCFO-1233zd/10% HFO-1234ze formulation. The Mist sprays with MBU actuators were in specification at 3 inches (7.6 cm) but not at 5 inches (13 cm) in both batches of testing. It was also found that the controls for the Mist Sprays were slightly out of specification as well. It is likely that the droplet size is smaller due to different chemical properties causing the droplets to disperse out before reaching the thermocouple.

### Example 4: Dispense Rates

Dispense rates for various vapocoolant compositions were determined.

REFERENCES: Modified Design P-27.3 Functionality Testing of Aerosol Skin Refrigerants; USP <601> Aerosols/Physical Tests: Delivery Rate.

EQUIPMENT: Water bath capable of maintaining a temperature of 25° C.; Analytical balance; Timer.

TEST SAMPLES—MATERIAL: 2(n=9) HCFO-1233zd 100%; 2(n=9) HCFO-1233zd 95%/HFO-1234ze 5%; 2(n=9) HCFO-1233zd 90%/HFO-1234ze 10%; 2(n=9) HCFO-1233zd 80%/HFO-1234ze 20%; 2(n=9) HCFO-1233zd 70%/HFO-1234ze 30%. HCFO-1233zd corresponded to Honeywell SOLSTICE® 1233zd(E). HFO-1234ze corresponded to Honeywell HFO-1234ze blowing agent.

TEST SAMPLES—COMPONENTS: 2(n=15) Medium Stream can; 2(n=15) Fine Stream can; 2(n=15) Mist can.

TEST SAMPLES—HFC CONTROLS: (n=4) Gebauer's PAIN EASE® Mist brand product (lot 1); (n=3) Gebauer's PAIN EASE® Mist brand product (lot 2); (n=4) Gebauer's SPRAY AND STRETCH® Fine Stream brand product (lot 1); (n=3) Gebauer's SPRAY AND STRETCH® Fine Stream brand product (lot 2); (n=7) Gebauer's PAIN EASE® Medium Stream brand product (lot 1).

METHODS: An initial 45 cans were sampled, 15 Medium Stream cans, 15 Fine Stream cans, and 15 Mist cans. The caps were removed from each can but the actuators remained assembled. Each can was weighed to the nearest 10 mg and recorded as W1. The cans were then encased in a plastic sleeve and immersed in a constant-temperature water bath at 25° C. for 30 minutes. The capacity for the water bath allowed 15 cans at a time. Each can was removed from the water bath and dried with a towel. The cans were then actuated for approximately 5 seconds. The exact time (T) was recorded and the cans returned back to the bath. This process was repeated for a total of three times. Each can was weighed and recorded as Wf. The average dispense rate was then calculated by subtracting (W1−Wf)/T.

This process was repeated with the HFC controls to determine a baseline. An initial 12 control cans were sampled, 4 HFC Medium Stream cans, 4 HFC Fine Stream cans, and 4 HFC Mist cans. Another batch of testing was performed following the same procedures for a total of 80 cans sampled and a total of 21 control cans sampled.

Functionality observations of the cans were also recorded. While performing each spray, the stream/mist was observed for continuous application, dripping, frosting, splitting or sputtering, and for determining whether the unit continued to spray when the actuation was stopped.

RESULTS: Results are provided in TABLE 4.

TABLE 4
Dispense rates of hydrofluoroolefin vapocoolant compositions comprising
HCFO-1233zd/HFO-1234ze and of HFC control compositions, from different actuator
types.
NMBU MEDIUM STREAM (0.43-0.73 g/s)

| | | 100%/ | 95%/ | 90%/ | 80%/ | 70%/ |
|---|---|---|---|---|---|---|
| SAMPLE | CONTROL | 0% | 5% | 10% | 20% | 30% |
| AVERAGE DISPENSE RATE (g/s) | 0.65 | 0.67 | 0.62 | 0.58 | 0.66 | 0.69 |
| STANDARD DEVIATION | 0.06 | 0.12 | 0.04 | 0.05 | 0.03 | 0.03 |
| MINIMUM DISPENSE RATE (g/s) | 0.46 | 0.52 | 0.56 | 0.52 | 0.62 | 0.65 |
| MAXIMUM DISPENSE RATE (g/s) | 0.71 | 0.79 | 0.67 | 0.65 | 0.70 | 0.72 |

NMBU FINE STREAM (0.17-0.47 g/s)

| | | 100%/ | 95%/ | 90%/ | 80%/ | 70%/ |
|---|---|---|---|---|---|---|
| SAMPLE | CONTROL | 0% | 5% | 10% | 20% | 30% |
| AVERAGE DISPENSE RATE (g/s) | 0.33 | 0.38 | 0.39 | 0.31 | 0.33 | 0.35 |
| STANDARD DEVIATION | 0.04 | 0.04 | 0.09 | 0.03 | 0.05 | 0.03 |
| MINIMUM DISPENSE RATE (g/s) | 0.24 | 0.33 | 0.23 | 0.27 | 0.24 | 0.30 |
| MAXIMUM DISPENSE RATE (g/s) | 0.36 | 0.44 | 0.47 | 0.35 | 0.39 | 0.39 |

MBU MIST (0.45-0.75 g/s)

| | | 100%/ | 95%/ | 90%/ | 80%/ | 70%/ |
|---|---|---|---|---|---|---|
| SAMPLE | CONTROL | 0% | 5% | 10% | 20% | 30% |
| AVERAGE DISPENSE RATE (g/s) | 0.63 | 0.75 | 0.68 | 0.75 | 0.69 | 0.73 |
| STANDARD DEVIATION | 0.62 | 0.03 | 0.04 | 0.05 | 0.02 | 0.04 |
| MINIMUM DISPENSE RATE (g/s) | 0.64 | 0.71 | 0.61 | 0.66 | 0.65 | 0.67 |
| MAXIMUM DISPENSE RATE (g/s) | 0.57 | 0.78 | 0.74 | 0.81 | 0.72 | 0.81 |

NMBU MIST (0.45-0.75 g/s)

| | 100%/ | 95%/ | 90%/ | 80%/ | 70%/ |
|---|---|---|---|---|---|
| SAMPLE | 0% | 5% | 10% | 20% | 30% |
| AVERAGE DISPENSE RATE (g/s) | 0.56 | 0.63 | 0.68 | 0.64 | 0.73 |
| STANDARD DEVIATION | 0.01 | 0.06 | 0.06 | 0.02 | 0.06 |
| MINIMUM DISPENSE RATE (g/s) | 0.54 | 0.56 | 0.59 | 0.59 | 0.65 |
| MAXIMUM DISPENSE RATE (g/s) | 0.57 | 0.71 | 0.71 | 0.69 | 0.81 |

DISCUSSION: All the averaged dispense rates for each type of can and for each formulation were within specification. A few individual sprays were out of specification. Results show similar dispense rates of the HFO formulations compared to the HFC controls. The Medium and Fine Stream cans have similar standard deviations compared to their controls, whereas the Mist cans exhibited more variability. Observations made during the dispense rate testing revealed some sputtering and splitting spray patterns seen mostly in the Fine Stream cans. This can be attributed to the faulty actuators. Replacement of these actuators resolved the spray pattern issues.

While this invention has been described in conjunction with the specific embodiments outlined above, it is evident that many alternatives, modifications and variations will be apparent to those skilled in the art. Accordingly, the preferred embodiments of the invention as set forth above are intended to be illustrative, not limiting. Various changes may be made without departing from the spirit and scope of the invention.

### Example 5: No Toxic Effects

REFERENCES: Acute Dermal Toxicity Study in Rats, NAMSA Test Report 17T_51965_03; United States Environmental Protection Agency, OPPTS Health Effects Test Guideline 870.1200.

METHODS: The test article corresponded to a hydrofluoroolefin vapocoolant composition of HCFO-1233zd and HFO-1234ze in an aluminum aerosol dispensing can. On the day of dosing, the animals were weighed, fur was clipped from the back and flanks from the shoulders to the hips, and the area of application was determined for each animal based on individual body weight. The test article was sprayed onto a substrate such as gauze for 5 seconds to clear away any debris from the spray nozzle. For one animal only, the test article container was weighed prior to dosing, and following application to the designated area, the test article container was weighed again in order to obtain an approximate volume administered. Each animal had test article applied to a single dermal site that was approximately 10% of its body surface area (also termed BSA). Ten percent BSA is considered a suitable exaggeration for preclinical safety testing. The test article was sprayed on the animal's back, holding the spray dispenser approximately 3 inches (7.6 cm) from the skin, to coat the designated area until the corresponding hydrofluoroolefin vapocoolant composition started to run off. The animal was held for approximately 1 minute, until the sprayed area appeared dry, then the animal was returned to its cage. This procedure was repeated for each animal. The day of dosing was day 0.

Detailed examinations for adverse reactions, clinical signs of disease, or abnormality were conducted at assignment, prior to dosing, at 1, 2.5 and 4 hours after dosing, and daily thereafter. These detailed observations involved animal handling and observation of the application site. Body weights were recorded prior to dosing (day 0), days 1, 2, 3, 7, and at termination on day 14. On day 14, detailed observations were performed and the animals were weighed. The animals were then euthanized by carbon dioxide inhalation. Following euthanasia, a macroscopic examination of the tissues and viscera (gross necropsy) were conducted. No tissues were collected.

RESULTS: The hydrofluoroolefin vapocoolant composition and aerosol dispensing container caused no toxic effects with living tissue or animal model patients. An acute dermal toxicity animal model was treated with the hydrofluoroolefin vapocoolant composition. Ten Sprague Dawley rats (5 male, 5 female) had the hydrofluoroolefin vapocoolant composition dispensed from an aerosol container, applied dermally over ˜10% of the body surface area. These animals were observed at 1, 2.5, and 4 hours following treatment and daily thereafter (Table 5). Body weights were measured prior to treatment, weekly, and at termination (Table 6). At 14 days, the rats were euthanized and a gross necropsy was performed (Table 7). There was no mortality during the course of the study, and at necropsy all animals appeared macroscopically normal (Table 7).

TABLE 5
Clinical observations from 5 male and 5 female Sprague Dawley rats
with hydrofluoroolefin vapocoolant composition dispensed from
an aerosol container covering 10% of body surface area.

| | Animal | Clinical and |
|---|---|---|
| Sex | Number | Application Site Observations |
| Male | 3611 | No findings |
| | 3612 | No findings |
| | 3613 | No findings |
| | 3614 | No findings |
| | 3615 | No findings |
| Female | 3617 | No findings |
| | 3618 | Days 7 through 9 - Dry |
| | | eschar over left shoulder area |
| | 3619 | No findings |
| | 3620 | No findings |
| | 3621 | No findings |

TABLE 6
Test animal body weights pretreatment, Day 1, 2, 3, 7, and 14 after
hydrofluoroolefin vapocoolant composition dispensed from an
aerosol container covering 10% of their body surface area.

| | | Body Weights (g) | | | | | |
|---|---|---|---|---|---|---|---|
| | Animal | | | | | | Day |
| Sex | Number | Pretreat | Day 1 | Day 2 | Day 3 | Day 7 | 14 |
| Male | 3611 | 333 | 323 | 324 | 330 | 331 | 346 |
| | 3612 | 340 | 335 | 343 | 344 | 360 | 383 |
| | 3613 | 345 | 338 | 345 | 346 | 356 | 376 |
| | 3614 | 350 | 349 | 358 | 360 | 377 | 405 |
| | 3615 | 355 | 343 | 353 | 352 | 365 | 401 |
| Female | 3617 | 229 | 225 | 219 | 222 | 228 | 240 |
| | 3618 | 220 | 220 | 221 | 216 | 236 | 271 |
| | 3619 | 222 | 213 | 213 | 213 | 218 | 235 |
| | 3620 | 224 | 215 | 215 | 216 | 220 | 233 |
| | 3621 | 220 | 220 | 222 | 216 | 222 | 238 |

TABLE 7
Macroscopic observations including clinical signs of disease after
hydrofluoroolefin vapocoolant composition dispensed from an
aerosol container covering 10% of their body surface area.

| | Animal | |
|---|---|---|
| Sex | Number | Macroscopic Observations |
| Male | 3611 | Macroscopically normal. |
| | 3612 | Macroscopically normal. |
| | 3613 | Macroscopically normal. |
| | 3614 | Macroscopically normal. |
| | 3615 | Macroscopically normal. |
| Female | 3617 | Macroscopically normal. |
| | 3618 | Macroscopically normal. |
| | 3619 | Macroscopically normal. |
| | 3620 | Macroscopically normal. |
| | 3621 | Macroscopically normal. |

DISCUSSION: The acute toxicity test data demonstrated that the hydrofluoroolefin vapocoolant composition dispensed from an aerosol container did not elicit any toxic effects. There were no clinical observations noted that appeared to be related to the administration of the hydrofluoroolefin vapocoolant composition from an aerosol dispensing package. There was no evidence of acute toxicity following the dermal application of the test article.

While this invention has been described in conjunction with the specific embodiments outlined above, it is evident that many alternatives, modifications and variations will be apparent to those skilled in the art. Accordingly, the preferred embodiments of the invention as set forth above are intended to be illustrative, not limiting. Various changes may be made without departing from the spirit and scope of the invention.

### INDUSTRIAL APPLICABILITY

The devices, compositions, and methods disclosed herein are useful for being administered topically to patients, e.g. human and/or animal patients, to treat pain and/or swelling by acting as a vapocoolant that cools surfaces of tissues of the patients.

1. A device for dispensing a hydrofluoroolefin vapocoolant composition as an aerosol, the device comprising:
an aerosol-dispensing container comprising an internal chamber, a valve, a dispensing hole, and an actuator; and
a hydrofluoroolefin vapocoolant composition comprising trans-1-chloro-3,3,3-trifluoropropene (HCFO-1233zd) at 70% to 100% (weight/weight) and, optionally, trans-1,3,3,3-tetrafluoroprop-1-ene (HFO-1234ze) at 0% to 30% (weight/weight);
wherein the hydrofluoroolefin vapocoolant composition is stored within the internal chamber, and the aerosol-dispensing container is configured so that depressing the actuator reversibly opens the valve to allow the hydrofluoroolefin vapocoolant composition to flow from the internal chamber, past the valve, and through the dispensing hole, thereby dispensing the hydrofluoroolefin vapocoolant composition from the aerosol-dispensing container.

2. The device of claim 1, wherein the hydrofluoroolefin vapocoolant composition comprises HCFO-1233zd at 70% to 99% (weight/weight) and HFO-1234ze at 1 to 30% (weight/weight).

3. The device of claim 2, wherein the hydrofluoroolefin vapocoolant composition consists essentially of HCFO-1233zd and HFO-1234ze.

4. The device of claim 2, wherein the hydrofluoroolefin vapocoolant composition comprises no further vapocoolant compounds.

5. The device of claim 1, wherein the hydrofluoroolefin vapocoolant composition comprises HCFO-1233zd at 75% to 95% (weight/weight) and HFO-1234ze at 5 to 25% (weight/weight).

6. The device of claim 1, wherein the aerosol-dispensing container is selected from the group consisting of a stainless steel aerosol can, an aluminum aerosol can, a plastic aerosol can, a polymer-based aerosol can, an aerosol bottle, a glass aerosol bottle, and a pressure container.

7. The device of claim 1, wherein the aerosol-dispensing container is configured to withstand pressures up to 300 psi (2.07 MPa).

8. The device of claim 1, wherein the actuator comprises an actuator insert comprising an orifice having a diameter of 0.007 to 0.016 inches (0.18 to 0.40 mm).

9. The device of claim 1, wherein the actuator is configured for dispensing the hydrofluoroolefin vapocoolant composition without breaking up flow of the hydrofluoroolefin vapocoolant composition.

10. The device of claim 1, wherein the actuator comprises a non-mechanical break up (NMBU) actuator.

11. A method of treating pain and/or swelling of a tissue of a patient by use of the device of claim 1, the method comprising a step of dispensing the hydrofluoroolefin vapocoolant composition from the device onto the tissue of the patient.

12. The method of claim 11, wherein the patient is a human or an animal.

13. The method of claim 11, wherein the treating comprises managing pain and/or swelling, controlling pain and/or swelling, and/or relieving pain and/or swelling.

14. The method of claim 11, wherein the dispensing of the hydrofluoroolefin vapocoolant composition from the device onto the tissue of the patient comprises applying the hydrofluoroolefin vapocoolant composition to the tissue of the patient directly by spray and/or indirectly through an applicator.

15. The method of claim 11, wherein the tissue comprises at least one of intact skin tissue, intact mucous membrane tissue, oral cavity tissue, nasal passage tissue, lip tissue, or minor open wound tissue.

16. The method of claim 11, wherein the pain and/or swelling of the tissue is due to at least one of a needle procedure, venipuncture, an intravenous catheter start, a cosmetic procedure, a procedure comprising incision and drainage of a small abscess, a minor surgical procedure, a procedure comprising lancing of a boil, a suturing procedure, a stapling procedure, a dental procedure, muscle pain, a scrape, an abrasion, myofascial pain, a hair removal procedure, a tattoo procedure, a medical tattoo procedure, chronic pain, viral infection pain, herpes sore pain, and a bruise.

17. The method of claim 11, wherein the pain and/or swelling of the tissue is due to at least one of swelling from a sports injury, swelling from overuse of a muscle, swelling from overuse of a joint, and swelling from arthritis.

18. The method of claim 17, wherein the actuator of the device is positioned 2 to 20 inches (5 to 51 cm) from the tissue during the step of dispensing.

19. The method of claim 17, wherein the step of dispensing is carried out for 2 to 15 seconds.

20. The method of claim 11, wherein the treating comprises use of the hydrofluoroolefin vapocoolant composition as a counterirritant in the management of at least one of myofascial pain, restricted motion, or muscle tension.

21. The method of claim 20, wherein the actuator of the device is positioned 6 to 24 inches (15 to 61 cm) from the tissue during the step of dispensing.

22. The method of claim 20, wherein the step of dispensing comprises directing a spray of the hydrofluoroolefin vapocoolant composition in parallel sweeps 0.3 to 2 inches (0.8 to 5 cm) apart at a rate of approximately 3 to 5 inches (8 to 13 cm) per second.

23. The method of claim 11, wherein the treating comprises use of the hydrofluoroolefin vapocoolant composition for identifying pulp viability of teeth.

24. The method of claim 11, wherein the treating comprises use of the hydrofluoroolefin vapocoolant composition for tracking the efficacy of an epidural block.

25. The method of claim 11, wherein the treating has no toxic or irritating effect on a living tissue of the patient.
